(19) United States
(12) Patent Application Publication
(10) Pub. No.: US 2006/0161055 A1
(43) Pub. Date: Jul. 20, 2006

(54) Probe design

(75) Inventors: Eliahu Pewzner, Modiin (IL); Assaf Deutsch, Moshav Tzafaria (IL); Yoram Blum, Givat Shmuel (IL); Avraham Mayevsky, Ramat-Gan (IL)

Correspondence Address: WOLF, BLOCK, SCHORR & SOLIS-COHEN LLP, 250 PARK AVENUE, NEW YORK, NY, 10177, US

(73) Assignee: CritiSense, Ltd., Givat Shmuel (IL)

(21) Appl. No.: 11/311,203

(22) Filed: Dec. 19, 2005

Related U.S. Application Data
(63) Continuation-in-part of application No. 10/508,232, filed on May 23, 2005.

(30) Foreign Application Priority Data
Mar. 20, 2002 (IL) 148795

(51) Int. Cl. A61B 5/00

(52) U.S. Cl. 600/310

(57) ABSTRACT

An optical probe, for acquiring measurements of material in a surface, the probe comprising: a probe body; at least one illuminating optical fiber that transmits light to a distal end thereof to illuminate a region of the surface and interact with the material; and at least one receiving optical fiber, positioned to receive light that has been transmitted by the illuminating fiber to the region and has interacted with the material, which received light is used for acquiring the measurements, the receiving fiber thereby being defined as associated with the illuminating fiber; wherein at least one of the fibers has a portion inside the probe body with a bend.

### RELATED APPLICATIONS

This application is a continuation-in-part of U.S. application Ser. No. 10/508,232, filed May 23, 2005, which is the US national phase of PCT application PCT/IL03/00188, filed Mar. 6, 2003 and published as WO 03/077746 on Sep. 25, 2003, which takes priority from Israel application IL 148795, filed Mar. 20, 2002.

### FIELD OF THE INVENTION

The field of the invention relates to optical probes for measuring parameters of body tissue.

### BACKGROUND OF THE INVENTION

Optical methods are useful for measuring a number of different parameters in body tissue, which are useful in assessing tissue vitality. Some of these methods are described in PCT publication WO 02/024048 and its US national phase published application 2004/0054270, to Pewzner and Mayevsky, as well as in U.S. Pat. Nos. 5,685,313 and 5,916,171, both to Mayevsky, and in references cited therein. The measured parameters include blood flow, which can be measured by a laser Doppler flowmeter, NADH and flavoprotein levels, both indicative of mitochondrial redox state, which can be measured by fluorescence, and blood volume and oxygenation state, which can be measured by reflectivity at different wavelengths. Knowing both the mitochondrial redox state and the oxygen supply rate by the blood provides more useful information about tissue vitality than either one of those pieces of information by itself, especially if they are both measured simultaneously in a same volume of tissue, by a single instrument.

Optical methods may also be used to measure many other parameters of medical interest, for example blood glucose levels in diabetics, described for example in U.S. Pat. No. 5,551,422 to Simonsen et al.

Systems that are used to make such optical measurements generally comprise a light source, “illuminating” optical fibers, “receiving” optical fibers, and a detector. The illuminating fibers carry light at one or more wavelengths from the light source to the surface of the body tissue that is being measured. The receiving fibers receive a portion of the light that has penetrated and been scattered by the tissue and carry the received light to the detector, which produces an electrical signal that can be recorded and analyzed. Optical fibers may be made of a variety of materials, including fused silica, and polymers such as poly(methyl methacrylate), PMMA. Polymer optical fibers (POF) are sometimes used in single-use medical probes, since they are much less expensive than silica fibers.

U.S. Pat. No. 5,916,171 and WO 02/024048 respectively describe probes for making optical measurements of tissue parameters in the brain, and in body tissue in general. Each of the probes shown in the title page illustrations has a long, thin probe body, with optical fibers running along the longitudinal axis of the probe body, which is oriented perpendicular to the surface of the tissue when the probe is used.

When long, flexible optical fibers connect a light source and detector to an optical probe body, for example to perform laser Doppler measurement of blood flow, motion of the flexible fibers may cause motion artifacts that introduce error into the measurement of blood flow. Such motion artifacts are described, for example, by R. J. Gush and T. A. King, “Investigation and improved performance of optical fiber probes in laser Doppler blood flow measurements,” Medical & Biological Engineering and Computing, July 1987. Motion artifacts in laser Doppler blood flow measurements may also be caused by inadvertent motion of the probe body along the surface of the tissue.

“Laser Doppler Probes,” a pamphlet published by Perimed A B, in Jarfalla, Sweden [retrieved 12-15-05], retrieved from the Internet <URL: http://www.perimed.se/p_Products/probeb14.pdf>, describes, on page 4, an integrating laser Doppler probe, Probe 413(313), in which values from each of seven probe tips are optically integrated into one output value, to improve reproducibility in areas with large spatial variation. This pamphlet also describes, on page 5, a microtip MT B500-2, comprising an optical fiber ending in an angled tip, which can be used with a laser Doppler probe system.

Scanning optical microscopy tips, for example the near-field microscopy tips manufactured by Nanonics Imaging, Ltd., in Jerusalem, Israel, may comprise a free end of an optical fiber with a 90 degree bend, tapered down to a sharp point with dimensions much smaller than the fiber diameter, and even smaller than a wavelength of light.

Optical fibers with black coatings are known, and are described, for example, in U.S. Pat. No. 6,026,207 to Reddy et al., and in references cited therein.

The above cited patents and other publications are incorporated herein by reference.

### SUMMARY OF THE INVENTION

An aspect of some embodiments of the invention relates to an improved optical probe for acquiring optical measurements of parameters that characterize material in a surface, such as the interior wall of a lumen in the body, or an outer or interior surface of any body organ. In an exemplary embodiment of the invention, one or more optical fibers have a bend inside a body of the probe. Optionally, the one or more fibers run axially along the body of the probe, and their distal portions are bent away from the axial direction so that their distal ends face the surface. As a result, the distal ends are oriented to efficiently transmit light to illuminate the surface and collect light scattered from the surface. Optionally, the bend in the fibers is sufficiently sharp so that the fibers can fit into a probe body that is less than 3 mm in diameter. Optionally, the radius of curvature of the bend is less than 5 times the fiber diameter. Optionally, the bend is sharp enough so that the light transmitted by the fiber is attenuated by at least 5% in going through the bend.

The probe may be particularly useful when the probe is to be oriented with the longitudinal axis parallel to the surface. For example, in a narrow lumen, or in any narrow space, there may not be room to position a long, narrow probe unless it is oriented with its longitudinal axis parallel to the surface. Orienting the probe with its longitudinal axis parallel to the surface may also be advantageous when holding the probe against an outer surface of a soft, smooth organ.

An aspect of some embodiments of the invention relates to providing an optical Doppler probe system for measuring blood flow in the body, for example microcirculatory blood flow, in which the effects of motion artifacts are ameliorated at least to some extent. For example, the system detects when blood flow data is affected by a motion artifact and discards that data, or informs a user that the data may be affected by motion artifacts, or corrects the data for the motion artifacts.

In some embodiments of the invention, the probe is adapted for use in the urethra, and comprises a probe body which fits into a urinary catheter. Such a probe remains in place for an extended period of time, and may be used to monitor tissue parameters continuously with relatively little inconvenience in addition to that suffered by a patient as a result of the presence of the catheter.

In an exemplary embodiment of the invention, the probe comprises at least two receiving fibers. In addition to a first “signal” receiving fiber that receives light that has been transmitted along the probe and been scattered from body tissue, there is a second, “monitoring” receiving fiber, coupled with the signal receiving fiber such that the two fibers move together. For example, the two fibers are bundled together in a flexible cable. The monitoring receiving fiber receives light that has been transmitted along the probe, optionally, to its distal end but that has not interacted with body tissue. The light in both signal and monitoring receiving fibers is subject to same motion artifacts if the fibers move. The light received by both the receiving fibers is analyzed to find an apparent Doppler shift indicative of a blood flow rate. If the light received by the monitoring fiber shows an apparent Doppler shift, then this indicates that the fibers are moving and causing motion artifacts, since the light in the monitoring fiber has not, in fact, interacted with body tissue. An apparent Doppler shift seen in light received by the signal fiber at a same time that light received by the monitoring fiber indicates motion artifacts is optionally disregarded, since the apparent Doppler shift is likely due to the motion artifacts.

In some embodiments of the invention, the light transmitted along the probe and received by both receiving fibers is carried by a single “illuminating” fiber from a light source, generally a laser or LED, to a region of the illuminating fiber near its distal end, which has a relatively sharp bend. At the bend, a portion of the light leaks out of the fiber, and is received by the monitoring fiber, without ever going into the body tissue. A remainder of the light propagates to the distal end of the illuminating fiber from where it exits the fiber and illuminates the body tissue. A portion of the illuminating light scatters from the body tissue and is received by the signal fiber.

An aspect of some embodiments of the invention concerns an optical probe, comprising a plurality of optical fibers characterized by reduced cross-talk between the fibers. Cross-talk may be a problem particularly for fibers formed from a polymer that are usually used in disposable optical probes, because they in general have higher numerical apertures than silica fibers. In addition, polymer optical fibers are often used without a buffer layer, which may make them more susceptible to cross-talk.

In an embodiment of the invention, a surface region of at least one of the fibers is coated with a light-blocking material that prevents light from leaking between the at least one fiber and another of the plurality of fibers. The light-blocking material is, for example, a black glue or paint that absorbs light, or a material that reflects light. Optionally, less than 50% of the length of the fiber is coated with the light-blocking material. In some embodiments of the invention, the light-blocking material is used substantially only on radial surfaces near the distal end of the at least one fiber. Light has a relatively enhanced tendency to scatter from the distal end of a fiber, especially if the end has a flat surface. In the absence of the light-blocking material, the scattered light may exit the fiber through its radial surface near the end and enter another fiber. Using the light-blocking material near the distal end of the fiber can therefore be particularly advantageous.

An aspect of some embodiments of the invention relates to an optical probe for acquiring measurements of material in a surface, for example body tissue in an internal or external surface of the body, in which a plurality of different signals are produced for measurements made at different regions of the surface. The signals are analyzed, and the analysis may make the measurements more reliable than if they were acquired from only one region. For example, if there are at least three illuminated regions, and a measurement of a parameter from a first region gives very different results than measurements of the same parameter from the other regions, then the first region may be an atypical region of the surface, and the measurements from the first region are optionally discarded. A region with a non-capillary blood vessel close to the surface, for example, may be atypical if the measurements comprise laser Doppler measurements of blood flow in capillaries. Fluorescence measurements of NADH or flavoprotein concentrations may also differ in different regions of an internal or external surface of the body. The measurements resulting from analyzing the plurality of different signals may be more reliable than if light received from the different regions were integrated to produce a single signal. Optionally, the different regions have centers that are at least about 3.5 mm apart, so that the light power illuminating the different regions does not have to be added together in determining the maximum permissible exposure of body tissue to the light.

There is thus provided, in accordance with an exemplary embodiment of the invention, an optical probe, for acquiring measurements of material in a surface, the probe comprising:

a probe body;

at least one illuminating optical fiber that transmits light to a distal end thereof to illuminate a region of the surface and interact with the material; and

at least one receiving optical fiber, positioned to receive light that has been transmitted by the illuminating fiber to the region and has interacted with the material, which received light is used for acquiring the measurements, the receiving fiber thereby being defined as associated with the illuminating fiber;

wherein at least one of the fibers has a portion inside the probe body with a bend.

Optionally, the probe body is less than 3 mm in diameter.

Optionally, the bend is sufficiently sharp so that light of a wavelength used for acquiring the measurements is attenuated by at least 5% when passing through the bend.

Optionally, the bend has a mean radius of curvature, over at least one 20 degree segment, of less than 5 times the fiber diameter.

In an embodiment of the invention, the probe body comprises a structure which holds a portion of said at least one of the fibers, including the bend, rigidly in place with respect to the probe body.

In an embodiment of the invention, the probe has a longitudinal axis, and the portion of the fiber inside the probe lies substantially along the longitudinal axis proximal to the bend, and the bend orients the distal end of the fiber to face away from the axis.

Optionally, the distal end faces along a direction more than 45 degrees from the longitudinal axis.

Optionally, the distal end faces along a direction more than 80 degrees from the longitudinal axis.

Optionally, the at least one illuminating fiber and the at least one receiving fiber both have portions that lie substantially along the longitudinal axis inside the probe body, and end in a bend that orients the distal end facing away from the axis.

Optionally, the distal ends face directions more than 45 degrees from the longitudinal axis.

Optionally, the distal ends face directions more than 80 degrees from the longitudinal axis.

There is further provided, in accordance with an exemplary embodiment of the invention, a method of acquiring optical data of material in a surface, the method comprising:

placing an optical probe according to an embodiment of the invention against the surface, with the longitudinal axis substantially parallel to the surface, and the distal ends of the at least one illuminating optical fiber and the at least one receiving optical fiber in optical contact with the surface;

illuminating a region of the surface with light through the at least one illuminating optical fiber; and

generating the data responsive to light received from the region of the surface by the at least one receiving optical fiber.

Optionally, placing the probe against the surface comprises holding the probe manually, without mechanically fixing the probe in place with respect to the surface.

Optionally, the surface comprises a surface of an internal organ of the body, the method also including:

Surgically exposing the internal organ; and

leaving the probe in place against the surface, to monitor the internal organ when is the organ is no longer exposed.

In an embodiment of the invention, the material is human or animal tissue and the surface is a wall of a lumen inside the human or animal.

Optionally, at least one of the optical fibers is a polymer optical fiber.

Optionally, the at least one receiving optical fibers comprise two receiving optical fibers, associated with one of the at least one illuminating optical fibers.

In embodiment of the invention, the at least one illuminating optical fiber comprises at least two illuminating optical fibers.

Optionally, the at least two illuminating optical fibers have distal ends the centers of which are between 2.5 and 5 mm apart.

Optionally, the at least two illuminating optical fibers have distal ends the centers of which are at least 3.5 mm apart.

Additionally or alternatively, the distal ends of the at least two illuminating optical fibers are more than 5 times as far apart as the penetrating distance in the material in the surface, of the most penetrating light of the illuminating light that interacts with the surface material.

Additionally or alternatively, the light transmitted by the at least two illuminating optical fibers is used to acquire measurements of a same parameter of the material, and the at least two illuminating optical fibers have distal ends spaced apart at a distance over which variations in said parameter are substantially uncorrelated.

Optionally, the center of the distal end of the at least one receiving optical fiber is located at a distance from the center of the distal end of the at least one illuminating optical fiber that it is associated with, equal to less than two times a penetrating distance, in the material in the wall, of the least penetrating light of the illuminating light that interacts with the material.

There is further provided, in accordance with an exemplary embodiment of the invention, a urinary catheter comprising a probe according to an embodiment of the invention, the catheter adapted so that the probe is positioned to acquire measurements of the wall of the urethra, when the catheter is in place in the urethra.

Optionally, the catheter comprises at least one opening in its side, through which a distal portion of the illuminating fiber and a distal portion of the receiving fiber extend, such that the illuminating fiber and receiving fiber are optically coupled with the wall of the urethra when the catheter is in place in the urethra.

Optionally, the bend in the fiber is machined out of a volume of the fiber material, and thereby has relatively low internal stress.

There is further provided, in accordance with an exemplary embodiment of the invention, a system comprising:

an optical probe according to an embodiment of the invention; and

a light source, coupled to the proximal end of the at least one illuminating optical fibers, which source produces the light for acquiring the measurements, between 315 nm and 525 nm.

There is further provided, in accordance with an exemplary embodiment of the invention, an optical probe, for acquiring measurements of a material, the probe comprising:

a plurality of optical fibers adapted for transmitting light to and from the material to acquire said measurements; and

a light-blocking material, covering at least a portion but less than 50% of at least one of the optical fibers, that reduces optical crosstalk between the fibers.

Optionally, the light-blocking material reduces optical crosstalk by absorbing light.

Alternatively or additionally, the light-blocking material reduces optical crosstalk by reflecting light.

Optionally, the light-blocking material mechanically couples said optical fiber to the probe or to another optical fiber or to both.

In an embodiment of the invention, the probe comprises a probe body having a longitudinal axis, and an optical fiber of the plurality of optical fibers has a portion that lies substantially along the longitudinal axis and ends in a bend that orients a distal end of the fiber facing away from the longitudinal axis, and the portion of the fiber covered by the light-blocking material is between the bend and the distal end.

There is further provided, in accordance with an exemplary embodiment of the invention, an optical probe system for measuring blood flow in a tissue region, the system comprising:

a first optical circuit that provides light that interacts with the tissue and generates a first signal indicative of the blood flow in the tissue region, responsive to the interacting light; and

a second optical circuit that generates a second signal that indicates when the first signal is affected by a motion artifact.

Optionally, the light is coherent, and the first signal indicates blood flow by a variance in Doppler shifts.

Optionally, the first optical circuit comprises an illuminating optical fiber that transmits the light to the tissue region and a receiving signal optical fiber that receives the light the interacts with the tissue.

Optionally, the second optical circuit comprises a receiving monitoring optical fiber that receives light that has not interacted with the tissue.

Optionally, the illuminating optical fiber has a bend, and the light received by the receiving monitoring optical fiber leaks out of the illuminating optical fiber at the bend.

Optionally, the receiving optical fibers are constrained to move together, so that motion of the receiving signal optical fiber which causes a motion artifact in the first optical circuit also causes a motion artifact in the second optical circuit.

Optionally, the second optical circuit also comprises an illuminating monitoring optical fiber, constrained to move with the illuminating optical fiber of the first optical circuit, which transmits the light received by the receiving monitoring optical cable.

In an embodiment of the invention, the system also comprises:

a light source that provides the light transmitted by the first optical circuit to the tissue region, and the light received by the second optical circuit; and

an adaptive filter, adapted to filter the first signal, using the second signal, to produce a filtered first signal with reduced light source noise compared to the unfiltered first signal.

Optionally, the system also comprises a filter, adapted to filter the first signal, using the second signal, to produce a filtered first signal with reduced motion artifacts compared to the unfiltered first signal.

There is further provided, in accordance with an exemplary embodiment of the invention, an optical probe for acquiring measurements of material in a surface, the probe comprising:

a plurality of illuminating optical fibers that transmit light to illuminate spatially separated regions of the surface and to interact with the material in the regions;

a set of at least one receiving optical fiber associated with each of the illuminating optical fibers, each receiving fiber positioned to receive at least a portion of the light that has interacted with the material in the region illuminated by the associated illuminating fiber; and

an interface to a detector for each region, to convert light received from each region to a separate signal.

There is further provided, in accordance with an exemplary embodiment of the system for acquiring optical measurements of material in a surface, the system comprising:

an optical probe according to an embodiment of the invention;

a detector for each set of receiving fibers, which converts light received from each region into a signal for the region; and

a controller adapted to analyze the signals to produce a local measurement result from each region, and to use the local measurement results to produce the measurement, disregarding or giving less weight to aberrant local measurement results.

### BRIEF DESCRIPTION OF THE DRAWINGS

Non-limiting examples of embodiments of the present invention are described below with reference to figures attached hereto and listed below. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1 shows a schematic view of a system including a probe for making optical measurements of tissue parameters, according to an exemplary embodiment of the invention;

FIG. 2A shows a schematic perspective view of a portion of the probe of FIG. 1, shown inserted into a urinary catheter;

FIG. 2B is a schematic cut-away view of the catheter shown in FIG. 2A, showing an axial cross-section of the catheter;

FIG. 2C schematically shows a detailed view of a portion of the catheter shown in FIG. 2A;

FIG. 3A shows a schematic side cross-sectional view of a portion of the probe and catheter shown in FIG. 2, inserted into the urethra, in accordance with an exemplary embodiment of the invention;

FIG. 3B shows a schematic axial cross-sectional view of a cable comprised in the probe shown in FIG. 3A;

FIG. 3C shows a schematic view of a surface of the probe shown in FIG. 3A which is in contact with the inside of the urethra in FIG. 3A, in accordance with an exemplary embodiment of the invention;

FIG. 3D shows a schematic perspective view showing parts of the probe shown in FIG. 3A, before assembly of the probe;

FIGS. 4A and 4B show schematic cross-sectional views of a portion of an optical probe configured for laser Doppler measurements of blood flow, according to two different exemplary embodiments of the invention;

FIG. 5 shows a schematic plot of signals generated from the probe in FIG. 4A in accordance with an exemplary embodiment of the invention;

FIG. 6 schematically shows a plot of spectra of the signals shown in FIG. 5, at different stages of signal processing, in accordance with an exemplary embodiment of the invention;

FIG. 7 schematically shows a block diagram of an adaptive filtering circuit for processing the signals shown in FIG. 5, according to an exemplary embodiment of the invention; and

FIGS. 8A and 8B show schematic cross-sectional views of a portion of an optical probe for measuring tissue parameters, according to an exemplary embodiment of the invention.

### DETAILED DESCRIPTION OF EXEMPLARY EMBODIMENTS

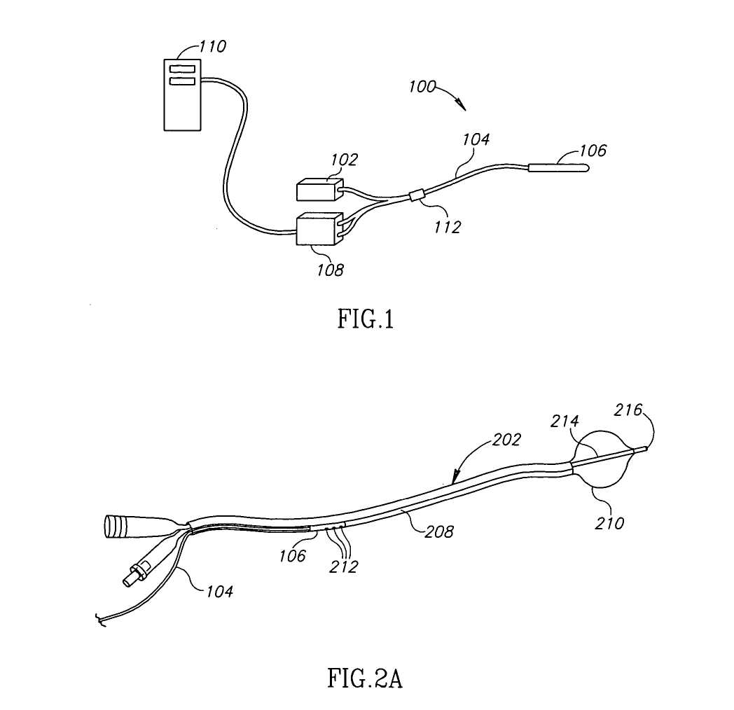

FIG. 1 shows a system 100 for making optical measurements of one or more tissue parameters, adapted for use in a narrow lumen such as the urethra, and/or adapted for use on a tissue surface of an organ such as the kidney or liver, exposed during surgery for example, or on the skin. For such a surface, particularly if it is soft and smooth, it may be difficult to hold the probe at a fixed position and angle of orientation with respect to the surface, in order to minimize or avoid motion artifacts. It may be easier to hold the probe in a fixed position and orientation by pressing a long surface of the probe against the surface of the organ.

A light source 102, comprising for example one or more lasers, LEDs, or lamps or any combination thereof, produces light at one or more wavelengths suitable for measuring one or more tissue parameters. Optionally the light source is filtered to eliminate unwanted wavelengths. The measured parameters include, for example, blood flow and tissue parameters mentioned above, using, for example, fluorescence or reflection. An optionally flexible cable 104, comprising one or more illuminating optical fibers, connects light source 102 to a probe body 106, which is adapted to be placed in the lumen and/or adapted to be placed on another tissue surface. As used herein, the term “probe” will generally refer to the probe body together with the cable. Light from the illuminating fibers illuminates the wall of the lumen or other tissue surface, and one or more receiving optical fibers in probe body 106 receive at their distal end or ends light scattered from tissue in the surface. The receiving fibers are, optionally, also housed in cable 104, and are connected at their proximal end or ends to a detection unit 108. Detection unit 108 generates one or more signals responsive to the light that it receives, which are transmitted to a controller 110, for example a computer, that analyzes the signals to determine the tissue parameters. Optionally, controller 110 also controls when light source 102 is turned on, and/or what wavelengths it produces and what power it operates at. Optionally, controller 110 also controls when detection unit 108 is turned on, and/or controls other aspects of detector unit 108.

The optical fibers may be any type of optical fiber known to the art, optionally a type that does not have high transmission losses for the wavelengths that are transmitted by the illuminating or receiving fibers. For example, for probes that use fluorescence to measure a tissue parameter, the illuminating light is often in the ultraviolet between 315 nm and 400 nm (the UVA band), or is visible light, for example between 400 and 525 nm. Suitable materials for fibers carrying light at these wavelengths include fused silica, particularly silica with a high OH content, which has good transmission properties in the UVA. Another suitable material is PMMA, which has sufficient UVA and blue transmission when the fibers are not too long, for example shorter than 10 meters.

Polymer optical fibers have some potential advantages over silica fibers. Polymer fibers are less expensive, typically by an order of magnitude, which may be important for disposable medical probes that are only used once, or a small number of times. Polymer fibers generally have a larger numerical aperture than silica fibers, which may be advantageous for use of a light source 102 that comprises a LED coupled directly to the fiber. If the illuminating fiber is silica, a more complicated and expensive coupling element may be needed between the fiber and the light source, or a relatively expensive light source may be needed. In addition polymer fibers can be bent quite sharply, with a radius of curvature comparable to the fiber diameter, while silica fibers may tend to develop cracks and eventually break if they are put under stress by being bent sharply. A fiber having a sharp bend that is not under high stress, and not prone to cracking, even if it is made of silica, can be machined from a volume of the silica or other fiber material, rather than by bending a fiber that is initially straight. However, such a process is generally expensive and may not be practical, particularly for a disposable probe. An effective “bend” may also be produced in a fiber, made of silica or other material, by coupling two straight segments of fiber to a reflecting element, but using such a method may also be too expensive to be practical.

Optionally, some or all of the optical fibers are housed in separate cables. Optionally, different components of light source 102, for example separate lasers generating different wavelengths of light, are housed in separate units connected through optical fibers to probe body 106. Optionally, detection unit 108 comprises two or more separate detectors, and each detector receives light from a different receiving fiber and generates signals responsive to the received light. Alternatively, a multi-wavelength signal in a single receiving fiber or single bundle of optical fibers is separated into discrete wavelengths, for example by a set of dichroic mirrors, and each wavelength is directed to a separate detector. Each detector optionally generates a signal corresponding to a different one of the tissue parameters. The different detectors need not be housed together in a single detection unit 108, as shown in FIG. 1, but optionally are housed in two or more separate units. In addition, separate controllers are optionally used to analyze different signals.

Optionally, cable 104 is coupled to detection unit 108 and/or to light source 102 through an optical connector 112, which contains an RF ID chip. Optionally, the RF ID chip communicates with controller 110, sending an RF signal that enables the probe by authorizing controller 110 to turn on light source 102 or detection unit 108, for example, or to analyze data from the probe. Optionally, the RF ID chip only sends such an authorization signal once, and if the probe stops being used, for example if it is disconnected from light source 102 or if light source 102 is turned off, then the probe cannot be enabled and used again, for example to ensure that the same probe is not re-used for different patients. Alternatively, the RF ID chip contains a time measuring element, such as a clock and a memory, or a capacitor which discharges through a resistor, which indicates for how long the probe has stopped being used. If the probe has not been stopped for too long a time, for example if the probe has been temporarily disconnected from a patient in an intensive care unit for so that the patient can undergo an MRI or CT scan, then the RF ID chip allows the probe to be used again. Optionally, instead of or in addition to using a passive RF ID chip for this purpose, an active chip, which is supplied with power, is used for this purpose.

If the probe is used to measure tissue parameters of an internal organ, for example during surgery or another medical procedure where the organ is exposed, the probe is optionally left in place inside the body for a period of time after the medical procedure. The probe can continue to monitor tissue parameters of the organ, and may for example be used to diagnose problems which arise after surgery.

Optionally, probe body 106 has a diameter at least twice as great as the diameter of the optical fibers which are inside it, or at least five times as great, or at least ten times as great. Optionally, probe body 106 has a length at least twice as great as the diameter of the optical fibers which are inside it, or at least five times as great, or at least ten times as great. Optionally, probe body 106 gives the optical fibers some additional stiffness or rigidity, beyond what the fibers would have by themselves. Optionally, probe body 106 helps give the distal end of one or more of the optical fibers a stable position and/or orientation with respect to the tissue and/or the distal end of one or more other fibers. Optionally, distal portions of the optical fibers inside probe body 106 are held rigidly in place by the probe body.

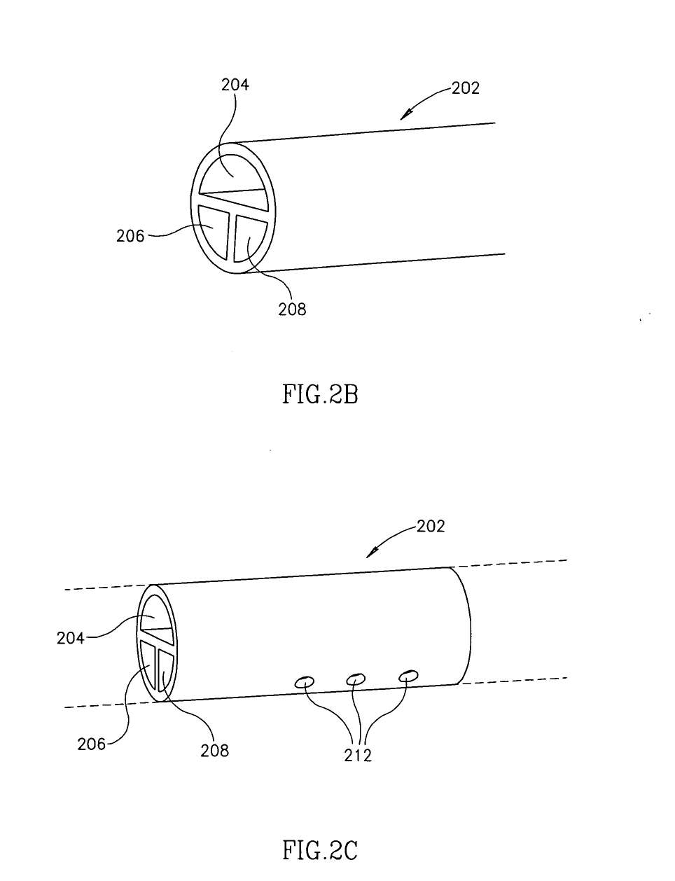

FIG. 2A schematically shows probe body 106, with a portion of cable 104, inserted into a urinary catheter 202, for example a Foley catheter made of silicone or latex. Probe body 106 and cable 104 are optionally sized so that they can be incorporated into an existing catheter, optionally without causing any change in the outer dimensions of the catheter. Urinary catheters are sometimes made with a probe lumen that can be fitted with a temperature probe. Probe body 106 and cable 104 are optionally sized so that they can be incorporated into the probe lumen of such a catheter, instead of the temperature probe, with no need for extensive changes in the catheter design. For example, probe body 106 is optionally less than 3 mm in diameter, or less than 2.5 mm in diameter, or less than 2 mm in diameter. Probe body 106 is optionally about 11.5 mm long and has a cross-section that is about 2.1 mm by 2.7 mm, and cable 104 optionally has a cross-section 1 mm wide.

Urinary catheter 202 optionally has a balloon 210 attached to a distal portion 214 of the catheter, which balloon is inserted into the bladder and inflated, in order to hold catheter 202 in place. The catheter optionally comprises three lumens, as shown in a more detailed view in FIG. 2B. A urinary lumen 204 carries urine out of the bladder. A balloon inflating lumen 206 carries a fluid, for example a saline solution, under pressure into balloon 210, to inflate the balloon. A probe lumen 208 is used for inserting the optical probe, or a temperature probe, into catheter 202. Lumens 204 and 206 are not visible inside catheter 202 in FIG. 2A, but the wall of lumen 208 is shown as if it were transparent, so that probe body 106 and cable 104 are visible inside lumen 208. Distal portion 214 of lumen 204 is shown extending through balloon 210, which is also shown as transparent in FIG. 2A. An opening 216 at the distal end of lumen 204, on the other side of balloon 210, is inside the bladder where it can collect urine, when catheter 202 is being used.

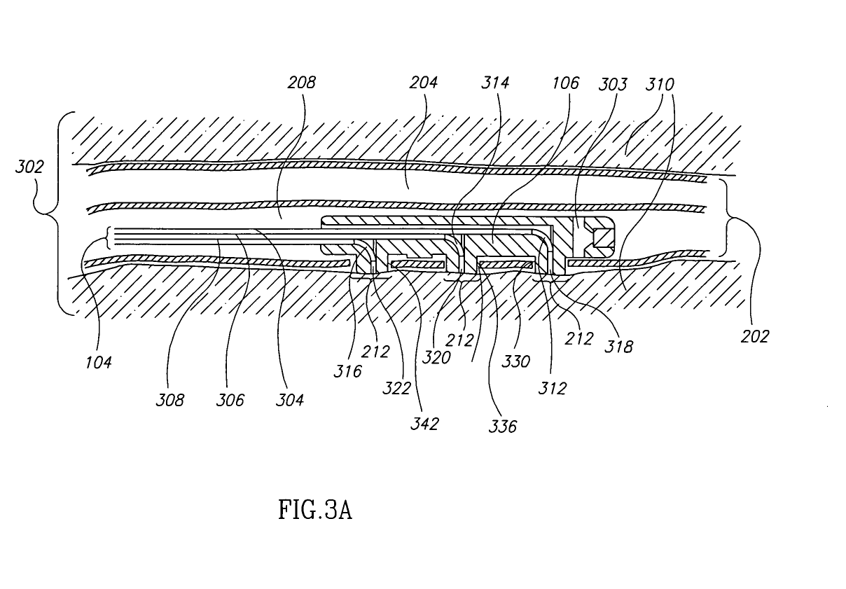

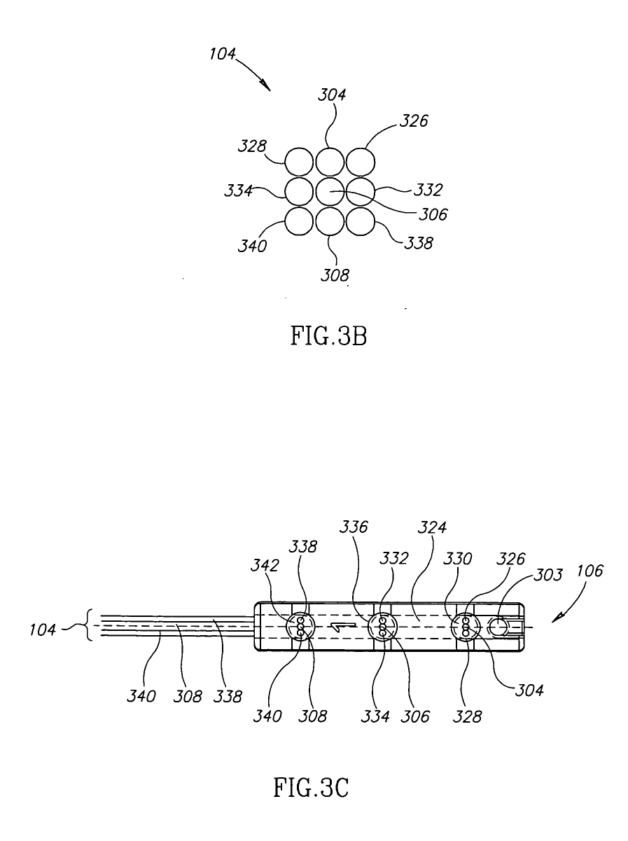

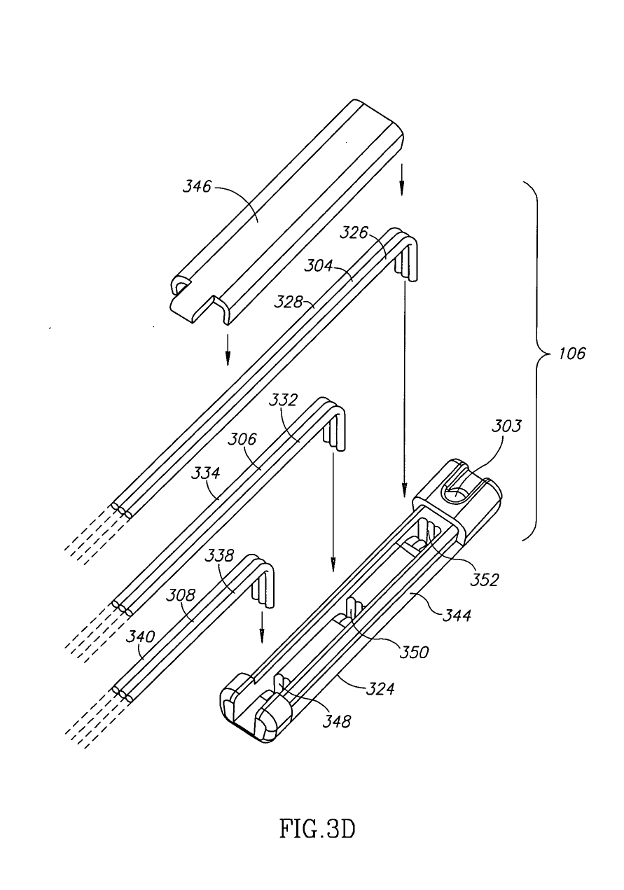

Optionally, there are one or more openings 212 in the wall of lumen 208, which are used by probe body 106 to view the tissue in the wall of the urethra. Openings 212 are shown in FIG. 2A, and in a more detailed view in FIG. 2C. Optionally, as shown in FIGS. 3A and 3C, probe head 106 has projections (three of them, labeled 330, 336 and 342, are shown in FIGS. 3A and 3C) which fit into openings 212, allowing the ends of the optical fibers to directly contact and/or optically couple to the wall of the urethra. The projections also optionally serve to hold probe 106 in place inside catheter 202.

Optionally, the portion of cable 104 inside lumen 208 comprises only the optical fibers, without an outer protective sheath holding them together, since the wall of lumen 208 serves to hold them together and protect them. Optionally, the portion of cable 104 outside catheter 202 has a protective sheath surrounding the optical fibers.

In some embodiments of the invention, probe body 106 is used in a lumen of the body other than the urethra, and may have different dimensions, such that the probe is adapted for insertion in the other lumen. A potential advantage of using probe body 106 having the dimensions noted above in the urethra is that, if the patient has a urinary catheter inserted for other reasons, probe body 106 may be kept inserted in the urethra with no additional discomfort or inconvenience to the patient, and used to monitor body tissue parameters continuously.

Optionally at least the portion of cable 104, inside lumen 208, is sufficiently flexible so that its presence inside lumen 208 does not substantially decrease the flexibility of catheter 202. Having such a flexible cable has the potential advantage that it does not make catheter 202 less comfortable for the patient than it would be without cable 104. Although probe body 106 is optionally rigid enough to make catheter 202 substantially less flexible at the location where probe body 106 is located, preferably probe head 106 is short enough so that it can be positioned in a straight portion of the urethra where catheter 202 does not have to bend. An example of a probe body and cable which will not affect patient comfort is the probe body described above, and the cable described below in FIGS. 3A-3C. This cable has a cross-section consisting of a 3×3 array of 0.25 mm diameter polymer optical fibers, and optionally is at least 1 meter long, or at least 1.5 meters long, or at least 2 meters long. Optionally, the cable is less than 10 meters long, or less than 4 meters long. If the cable is too short, and its proximal end is attached to the light source and detection unit, it may exert axial or lateral forces on the catheter which would cause patient discomfort. If the cable is too long, it may absorb a significant fraction of UVA or blue light.

FIG. 3A schematically shows a side cross-sectional view of probe body 106, inserted inside urinary catheter 202, positioned inside a urethra 302, in accordance with an embodiment of the invention. Optionally, there is a towing hole 303 at the distal end of probe body 106, used to pull probe body 106 into position in lumen 208, when catheter 202 is assembled. Cable 104 optionally comprises nine optical fibers, arranged in a 3×3 array, three groups of three fibers each. A cross-sectional view of cable 104, showing the 3×3 array of fibers, is shown in FIG. 3B, described below. Optionally, the fibers in a same group are coplanar. Optionally, the planes of fibers in different groups are parallel. In FIG. 3A, only one of the groups, comprising fibers 304, 306, and 308, is shown. Each fiber has, for example, a circular cross-section of diameter about 0.25 mm, allowing the 3×3 array of fibers to fit comfortably into the I mm square cross-section of cable 104.

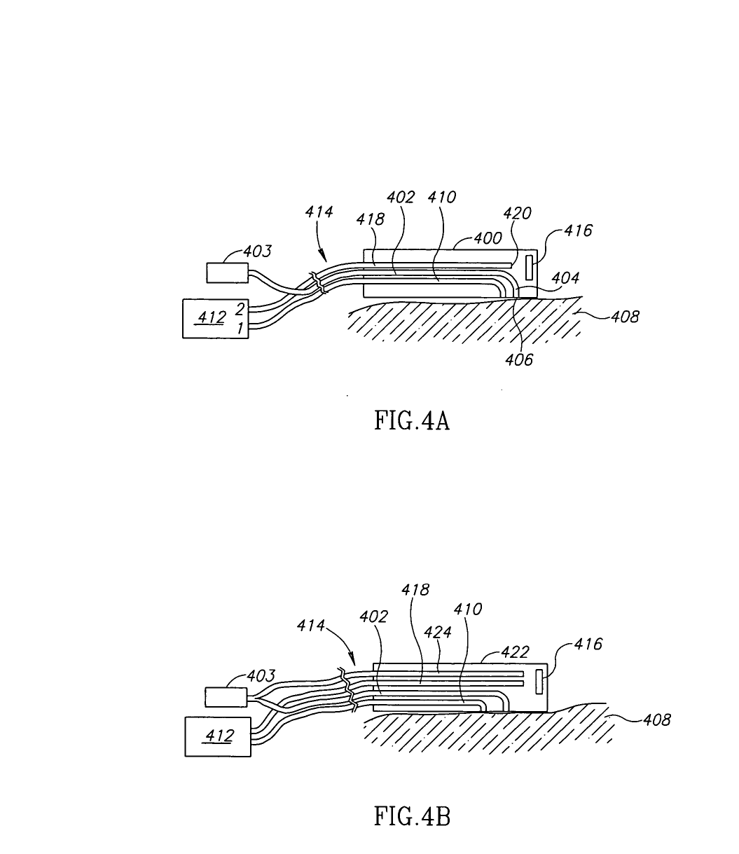

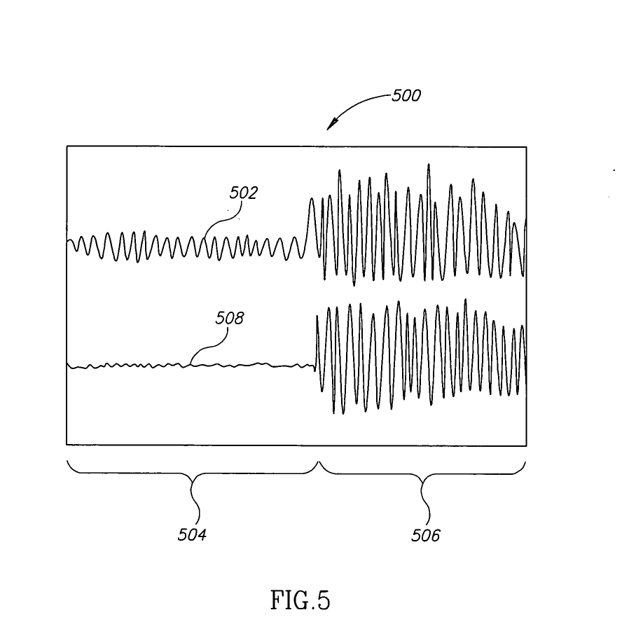

Each of the optical fibers in probe body 106 optionally has, near its distal end, an optionally 90 degree bend of relatively small radius of curvature, for example a radius of curvature equal to 0.7 mm which is 2.7 times its diameter, or a radius of curvature of 0.5 mm, or 1 mm, or a smaller or larger or intermediate value. If the radius of curvature is not uniform throughout the bend, then the numbers given here for radius of curvature optionally apply to the minimum local radius of curvature, or to the minimum radius of curvature averaged over any 20 degree segment of the bend, or averaged over any 45 degree segment of the bend. Optionally, the radius of curvature is less than 5 times the fiber diameter, or less than 4 times the fiber diameter, or less than 3 times the fiber diameter. Optionally, the bend is sufficiently sharp so that a significant fraction of the light transmitted by the fiber leaks out at the bend, at the wavelength or wavelengths used for measuring the tissue parameters. Optionally, the attenuation of the light in the bend is at least 5%, or at least 10%, or at least 20%. It is potentially advantageous for the bend to be sharp enough for some light to leak out, since, as will be described below in the description of FIG. 4A, the light that leaks out can be used to detect motion artifacts in laser Doppler measurements of blood flow. Having a bend with smaller radius of curvature also is potentially advantageous because it allows the probe body to have smaller diameter, for example less than 3 mm, and to fit into smaller spaces, such as probe lumen 208 of catheter 202. But if the bend is too sharp and too much light leaks out, there will be less light power available for measuring the tissue parameters, and the signal to noise ratio may be lower. In some embodiments of the invention, where having a small probe body diameter and having a high signal to noise ratio are both important, the radius of curvature of the bends is made as small as possible, subject to a constraint that no more than a moderate fraction of the light leaks out of the bends, for example no more than 20%, or no more than 40%.

Although the bend need not be 90 degrees, it is optionally close to 90 degrees, for example at least 80 degrees, or at least 70 degrees, or it is at least 45 degrees. Each fiber terminates optionally in a short straight section after the bend, oriented substantially perpendicular to the longitudinal axis of probe head 106 (oriented in a horizontal direction in FIG. 3A). As a result, when probe body 106 is inserted into urethra 302 the straight section of the fiber after the bend is substantially perpendicular to the wall of the urethra. For example, fibers 304, 306 and 308 respectively have bends 312, 314, and 316, and short straight sections 318, 320, and 322, which are oriented substantially perpendicular to the wall 310 of the urethra and positioned so that their ends are adjacent to wall 310 of the urethra when probe body 106 is inserted in the urethra.

The three fibers in each of the other two groups in the 3×3 array in cable 104 optionally have configurations near their distal ends similar to fibers 304, 306, and 308. That is to say, each fiber optionally has a 90 degree bend of optionally 0.7 mm radius of curvature, followed by a short straight section at its end, oriented perpendicular to urethra wall 310, but in a plane behind or in front of the plane shown in FIG. 3A. One of the other groups, located in a plane in front of the plane shown in FIG. 3A, consists of a fiber 326 in front of fiber 304, a fiber 332 in front of fiber 306, and a fiber 338 in front of fiber 308. The third group, located in a plane behind the plane shown in FIG. 3A, consists of a fiber 328 behind fiber 304, a fiber 334 behind fiber 306, and a fiber 340 behind fiber 308. FIG. 3B is a view of an axial cross-section of cable 104, showing the 3×3 array of fibers.

The ends of all nine fibers, seen head on, are visible in FIG. 3C, which shows an external view of a face 324 of probe body 106; face 324 is the face at the bottom of probe body 106, facing urethra wall 310, in FIG. 3A. Fibers 304, 326 and 328, which define a first row of the 3×3 array of fibers in cable 104, optionally have their ends located close together in projection 330, which extends a short distance out from face 324 of probe body 106. Similarly, fibers 306, 332 and 334, which define a second row of the 3×3 array, have their ends located in projection 336, and fibers 308, 338 and 340, which define a third row of the 3×3 array, have their ends located in projection 342. Projections 330, 336, and 342 position the ends of the fibers adjacent to wall 310 of the urethra, when probe body 106 is inserted into the urethra. The fibers in the different projections optionally are used for measuring different tissue parameters, or for measuring the same tissue parameters at different locations, for example to increase the signal to noise ratio and/or to increase the reliability of the measurements.

FIG. 3D schematically shows an exploded view of probe head 106, illustrating how probe head 106 and the optical fibers are assembled, according to an exemplary embodiment of the invention. A micro-plastic structure 344, with tow hole 303, constitutes the lower part of the probe shown in FIG. 3A. Micro-plastic structure 344 holds the optical fibers rigidly in place relative to each other and to the probe head, when the probe head is assembled. Structure 344 also optionally keeps the bends in the fibers, including bends 312, 314, and 316, fixed in shape. Surface 324 of probe 106, shown face on in FIG. 3C, is a lower surface of structure 344, and is hidden in FIG. 3D except at its edge. There is also a plastic cover 346, which is the upper part of probe 106 shown in FIG. 3A. When probe head 106 is assembled, fibers 340, 308, and 338 are first laid down in structure 344, with their bent end portions going down through grooves 348 in FIG. 3D, and ending in projection 342. Projection 342, like projections 336 and 330, is hidden in FIG. 3D but visible in FIGS. 3A and 3C. Fibers 334, 306 and 332 are then laid down on top of fibers 340, 308, and 338, with the bent end portions of fibers 334, 306, and 332 going down through grooves 350 in structure 344, and ending in projection 336. Next, fibers 328, 304, and 326 are laid down on top of fibers 334, 306, and 332, with the bent end portions of fibers 328, 304, and 326 going down through grooves 352 in structure 344, ending in projection 330. Optionally, when any of the fibers is laid down, glue is used to hold it in place. Finally, cover 346 is attached to the top of structure 344, locking the fibers into place inside probe head 106. Optionally, cover 346 is glued to structure 344, and/or to the tops of fibers 328, 304, and 326, and/or cover 346 snaps into place on top of structure 344. Optionally, structure 344 and cover 346 are each rigid, and are joined rigidly together, so that probe body 106 rigidly maintains its shape. This rigidity has the potential advantage that it may keep the distal ends of the optical fibers in fixed positions and orientations relative to probe body 106, and hence relative to the body tissue. The rigidity may also tend to prevent motion artifacts caused by motion of the fibers within the probe head. Alternatively, probe body 106 is malleable, which has the potential advantage that it can be adjusted to be used on surfaces of different shapes or degrees of curvature, for example.

Each of the nine fibers may be used as an illuminating fiber, carrying light from light source 102 (in FIG. 1) to probe body 106, where it illuminates the tissue in urethra wall 310, or as a receiving fiber, collecting light scattered from the tissue in urethra wall 310, and carrying it to detector 108 (in FIG. 1). In some embodiments of the invention, one or more fibers may serve both as an illuminating and a receiving fiber. Optionally, different fibers are used to carry different wavelengths of light, and/or to carry light that is used for measuring different tissue parameters. Optionally, some fibers carry light of more than one wavelength, and/or light that is used for measuring more than one tissue parameter.

In some embodiments of the invention, there are more than nine fibers, or fewer than nine fibers, and/or the fibers are arranged in cable 104 a different configuration than a 3×3 array. Having a larger number of fibers provides opportunities for conveying more signals and/or measuring more body parameters, using a separate fiber for each measurement. Using a separate fiber for each measurement may result in less interference between different measurements than if the same fiber is used for more than one measurement. Having a larger number of fibers also allows the same parameter to be measured at more locations, which may increase the reliability of the measurements. However, for given cable dimensions and probe dimensions possibly constrained by space available in the urethra or other lumen, or in the catheter, having fewer fibers allows each fiber to have a larger cross-section, and hence to convey more optical power for illuminating body tissue Conveying more optical power may allow a body parameter to be measured more quickly, and/or with higher signal to noise ratio. On the other hand, using fibers of greater diameter, for a given radius of curvature at the bends, may result in more light leaking out of the fibers at the bends. The radius of curvature at the bends may also be constrained by the space available in the urethra or other lumen or narrow space, or the space available in the catheter.

In an exemplary embodiment of the invention, fibers 304, 306, and 308, in the centers of projections 330, 336 and 342 respectively are used as illuminating fibers, and fibers 326, 328, 332, 334, 338 and 340, at the edges of projections 330, 336, and 342, are used as receiving fibers. Optionally, within each projection, the two receiving fibers are associated with the illuminating fiber in that projection. A receiving fiber is defined herein as “associated with” an illuminating fiber if the receiving fiber receives light, for measuring a tissue parameter, which was transmitted to the body tissue by the illuminating fiber and has interacted with the body tissue. The interaction may comprise scattering, for example, and may comprise being absorbed and re-emitted at a different wavelength (fluorescence).

In some embodiments of the invention, there is only one receiving fiber associated with each illuminating fiber, or there are three or more receiving fibers associated with each illuminating fiber, or there are sets of two or more illuminating fibers associated with the same one or more receiving fibers. In some embodiments of the invention there are only one or two sets of illuminating fibers and associated receiving fibers, or there are four or more sets of illuminating fibers associated with receiving fibers. In some embodiments of the invention, different sets of fibers, for measuring tissue parameters at different locations, have different numbers of receiving fibers or different numbers of illuminating fibers in them. In these embodiments of the invention, instead of a 3×3 array of fibers there may be a rectangular array of fibers in which the number of rows and/or the number of columns is different from 3, for example 2×2, 2×3, 3×2, 1×2, 3×1, or 4×3, or the fibers are not arranged in a rectangular array at all.

Optionally, illuminating light used for measuring two different tissue parameters, whether the light is a same wavelength or different wavelengths, is carried in a same illuminating fiber. The two receiving fibers adjacent to that illuminating fiber in the same projection are optionally each used for receiving light for measuring both of the two tissue parameters. In this case, the light from each receiving fiber is optionally split between two detectors, and each detector has a filter which admits light of the wavelength it is detecting. Alternatively, each receiving fiber is used for receiving light for measuring a different one of the two tissue parameters. However, using each receiving fiber to measure both parameters has the potential advantage that both parameters may be measured in the same or nearly the same tissue element, optionally at the same time. This arrangement may provide a better indication of the physiological state of the tissue than measuring the two tissue parameters in different tissue elements that are further apart. For example, blood flow and NADH are measured in nearly the same tissue element at the same time.

Optionally, the distance between the center of the distal end of an illuminating fiber, and the center of the distal end of a receiving fiber that receives light transmitted to the tissue by the illuminating fiber, is comparable to the penetration depth of the light in the tissue. For example, the distance is between 1 and 2 times the penetration depth. Optionally, the fiber diameter is as great or almost as great as the distance between the centers of the distal ends of the fibers, so that the two fibers are touching or nearly touching. In the case of UVA or blue light, in some kinds of body tissue, the penetration depth is about 0.2 mm, and the distance is optionally between 0.2 and 0.4 mm. Making the distance and the fiber diameter within this range, or close to this range, has the potential advantages that the received light power is about as great as possible, for a given illuminating light intensity, and the light power is used reasonably efficiently.

In an exemplary embodiment of the invention, the centers of the distal ends of illuminating fibers 304, 306, and 308 are spaced apart by a distance greater than about 2.5 mm. Optionally, they are spaced apart by a distance less than about 5 mm. Optionally, they are spaced apart by a distance between 2.5 mm and 5 mm. Optionally, they are spaced apart by about 3.5 mm. A spacing of at least 3.5 mm has a potential advantage due to the fact that, according to laser safety standards such as IEC60825-1, the maximum permissible exposure (MPE) of body tissue to laser light is based on the power deposited within an limiting aperture of diameter 3.5 mm. With the fibers spaced at least 3.5 mm apart, the power of light coming from different fibers is not combined in calculating the MPE. The maximum power can be used in each illuminating fiber, resulting in a higher signal to noise ratio and a more accurate measurement of tissue parameters. A potential advantage of not spacing the ends of the illuminating fibers more than 3.5 mm apart is that the different illuminating fibers can measure tissue parameters in tissue elements that are not too far apart, which may provide a more accurate indication of physiological state of the tissue than if the tissue elements were further apart. Alternatively, a different spacing between illuminating fibers may be used, and may be advantageous. For example, in some cases the advantages of making measurements in tissue elements that are closer together may outweigh the disadvantages of using lower power.

A further potential advantage of having at least two or at least three illuminating optical fibers, with distal ends spaced not too close together, is that results of the measurements may be more reliable, because there are multiple sensing regions. For example, if one of the illuminating optical fibers happens to illuminate a blood vessel substantially larger than a capillary, then the results of the measurements from that illuminating fiber may not be typical. The blood flow rate in a larger blood vessel, for example, is generally greater than the blood flow rate in capillaries. The concentration of NADH and flavoproteins in cells may be different at different locations. Two illuminating optical fibers that provide different measurement results indicate that the results from one of the illuminating fibers may be aberrant. If there are three or more illuminating optical fibers illuminating different sensing regions, and one of them gives very different measurement results, while the other illuminating fibers give measurement results that are consistent with each other, then this in general indicates that the results provided by the one fiber are aberrant. Optionally, controller 110 analyzes signals generated by detection unit 108 to produce local measurement results for each of the sensing regions, and optionally produces an integrated measurement result, disregarding, or giving less weight to, the local measurement results that are aberrant. It should be noted that this kind of analysis of the signals is possible if the receiving fibers from each sensing region connect to separate detectors, which produce separate signals, and this is a potential advantage of using separate detectors for each sensing region. Alternatively, light received from different sensing regions is fed to a single detector, which produces a single signal which is an average of what the signals would be from the different sensor regions, for example.

Optionally, a distance between different illuminating optical fibers is at least a few times greater than the penetrating distance of the light used for the measurements, for example at least five times as great as the penetrating distance for the most penetrating light used for the measurements. This ensures that the sensing regions illuminated by the different illuminating optical fibers effectively do not overlap. Optionally, a distance between different illuminating optical fibers is great enough so that variations in the tissue parameter being measured are substantially uncorrelated over that distance. For example, the correlation in the variations over that distance is less than 0.2, or less than 0.1. Then, if one of the illuminating fibers illuminates an atypical location for that tissue parameter, the other illuminating fiber or fibers will often illuminate more typical locations.

FIG. 4A schematically shows a probe body 400 used to acquire laser Doppler measurements of blood flow in body tissue, in accordance with an embodiment of the invention. In probe 400, an illuminating optical fiber 402 carries light from a laser 403, and has a relatively sharp optionally 90 degree bend 404 near its distal end 406, similar to the optical fibers in probe body 106 shown in FIG. 3A. Distal end 406 of fiber 402 is oriented substantially perpendicular to the axial dimension of probe body 400 so that when the probe is inserted into a lumen such as the urethra, or when the probe is placed against any tissue surface 408, distal end 406 is directed toward tissue surface 408, and the light carried by fiber 402 illuminates the tissue surface. In particular, light from fiber 402 illuminates red blood cells in capillaries in tissue surface 408. Light scattered from the tissue surface and the red blood cells, is received by a receiving signal optical fiber 410, which has its distal end adjacent to the illuminated region of surface 408. The scattered light is carried back to a first detector of a detection unit 412, which analyzes the scattered light to determine an average blood flow rate in the illuminated region. The blood flow rate is determined by measuring a level of fluctuations in the intensity of the light received by the detector, which level depends on a spread in Doppler frequency shifts of the light scattered from the moving red blood cells. An algorithm for finding blood flow rate from the intensity fluctuations in the scattered laser light is given, for example, by M. D. Stern, Nature, Vol. 254, Mar. 6, 1975, the disclosure of which is incorporated herein by reference.

Illuminating fiber 402 and signal fiber 410 are optionally bundled together in a flexible cable 414, similar to cable 104 in FIG. 1. Curvature of a flexible optical fiber generally produces a speckle pattern in the laser light, over the cross-section of each of the optical fibers, and the details of the speckle pattern depend on the curvature of the fiber over its length. If the cable moves and its curvature changes, for example due to mechanical vibrations produced by equipment in the vicinity, then the speckle pattern of the light received by the detector in general changes. The changing speckle pattern may produce intensity fluctuations that look similar to the intensity fluctuations produced by blood flow in the illuminated body tissue of surface 408. This may give rise to a motion artifact in the blood flow rate calculated from the light received by the detector. Although motion artifacts can also arise from motion of the probe body relative to the tissue, motion artifacts from that cause are likely to be less important in the case of a probe stably embedded in a urinary catheter which is stably positioned in the urethra, for example anchored by a balloon.

In order to distinguish a motion artifact from the real blood flow rate, in accordance with an embodiment of the invention, light leaking out of bend 404 of illuminating fiber 402 is used to illuminate a surface 416 adjacent to bend 404, inside probe 400. Surface 416 is, for example, a diffuse white opaque surface, optionally fixed rigidly in place with respect to bend 404. Surface 416 need not be part of an element of probe 400 included just for this purpose, but is optionally a structural part of probe 400. A light diffusing plastic, such as the acetal resin sold by DuPont under the brand name Delrin®, is satisfactory for both purposes. A receiving monitoring fiber 418 has its distal end 420 inside probe 400, adjacent to surface 416, and receives light from fiber 402 scattered from surface 416. Distal end 420 of fiber 418 is also optionally fixed rigidly in place with respect to surface 416 and bend 404. Fiber 418 is bundled with fibers 402 and 410, in cable 414. The light received by monitoring fiber 418 is carried back to a second detector of detection unit 412, and the fluctuations in the light received by the second detector are analyzed to calculate what the “blood flow rate” would be if the light received by the second channel were light from a laser Doppler measurement. Because surface 416 is not moving with respect to the distal regions of fibers 402 and 418, an analysis of the fluctuations of the light received by the second detector should show a very low fluctuation level, corresponding to zero “blood flow rate,” in the absence of motion artifacts.

If there is only a very low level of fluctuations seen in the light received by monitoring fiber 418, then any fluctuations in the light received by signal fiber 410 are accepted as indicating a real blood flow rate. If cable 414 is moving and changing its curvature, however, then both signal fiber 410 and monitoring fiber 418, will change their curvature, and will produce changing speckle patterns, resulting in motion artifact fluctuations in the light intensity received by both detectors. If the calculated “blood flow rate” is similar for the light received by signal fiber 410 and the light received by monitoring fiber 418, then the “blood flow rate” calculated from the light received by signal fiber 410 is likely due largely to motion artifacts, and is optionally disregarded.

FIG. 4B schematically shows a probe body 422 having an alternative design which allows motion artifacts to be distinguished from real blood flow rate in laser Doppler measurements of blood flow rate in accordance with an embodiment of the invention. Instead of relying on light leaking out of bend 404 in fiber 402 to detect motion artifacts, a second illuminating fiber 424, bundled together with fiber 402 in cable 414, ends inside probe body 422, and is used to illuminate surface 416. As in FIG. 4A, light scattered from surface 416 is received by monitoring fiber 418, which is bundled together with signal fiber 410 in cable 414, and carries light to the second detector. As in FIG. 4A, light from illuminating fiber 402 is scattered from body tissue, including moving red blood cells, and received by fiber 410, where it is carried to the first detector. Because the light received by the second detector follows the same path through the possibly moving cable as the light received by the first detector, it is expected to be subject to the same motion artifacts as light received from signal fiber 410, and can be used to determine when the blood flow rate determined from the first detector signal is reliable.

FIG. 5 schematically shows a graph 500 of a signal 502, representing the intensity of light received by signal fiber 410 as a function of time, during a time interval 504 when there are essentially no motion artifacts due to motion of cable 414, and during a time interval 506 when there are large motion artifacts. Graph 500 also includes a signal 508, representing the intensity of light received by monitoring fiber 418 during the same two time intervals.

During interval 504, signal 502 shows a moderate level of fluctuations, due to the Doppler shift produced in the light when it scatters from moving red blood cells in the body tissue of surface 408. Signal 508 is nearly flat and contains only electronic and laser fluctuations noises during interval 504, because the light received by fiber 418 did not scatter from body tissue.

During interval 506, signal 502 exhibits large fluctuations, due primarily to the motion artifacts caused movement of cable 414. Monitoring fiber 418 undergoes the same changes, and the light received by both fibers 410 and 418 is propagated through probe 400 by illuminating fiber 402. The light received by the second detector from fiber 418 is thus expected to be subject to the same motion artifacts as the light received by the first detector from fiber 410. During time interval 506, when cable 414 is moving, signal 508 has a high level of fluctuations, similar to signal 502.

In order to eliminate motion artifacts from the blood flow data determined from signal 502, the calculated blood flow data is optionally disregarded when signals generated responsive to light from monitoring fiber 418 exhibit fluctuations indicative of motion artifacts. Alternatively, possibly depending on the level of fluctuations seen in the light from monitoring fiber 418, the blood flow data is not disregarded, but is reported to a user of the probe as possibly being affected by motion artifacts. Alternatively, as will be described below, the blood flow data is adjusted, to reduce the effects of motion artifacts.

The calculated blood flow data is disregarded, reported as suspicious, or adjusted, for example, when the motion artifact level, as indicated by the fluctuation level of signal 508, is more than a predefined level. This predefined level may be a function of the measured blood flow measurement, for example a particular percentage of the fluctuation level of signal 502. For example, if the fluctuation level of signal 508 indicates a blood flow level that is more than 10% of the blood flow level calculated by the fluctuation level of signal 502, then the calculated blood flow rate is disregarded, reported as suspicious, or adjusted.

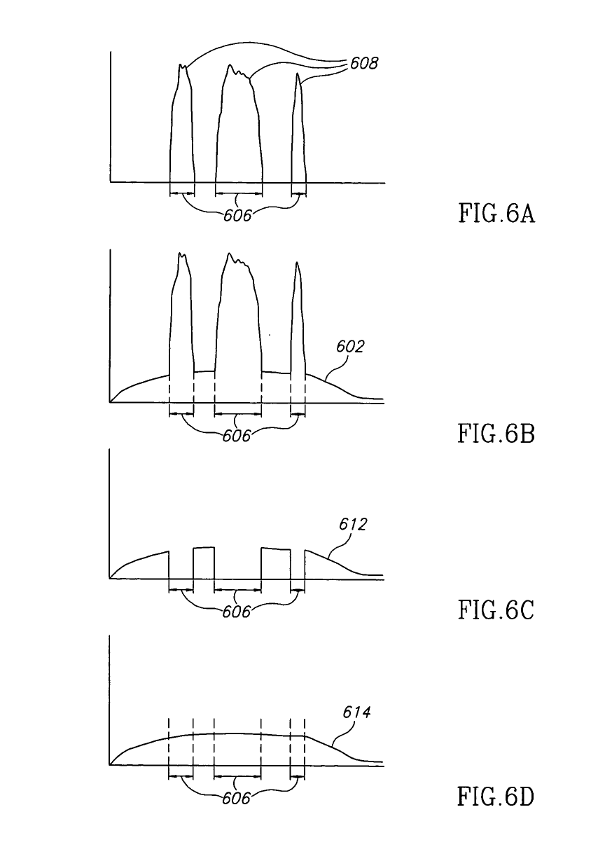

In some embodiments of the invention, the fluctuation level seen in signal 508 from monitoring fiber 418 is used to make adjustments in signal 502, to find a blood flow rate corrected for motion artifacts. This is optionally done, for example, by the filtering method shown in FIGS. 6A-6D. The fluctuations in light intensity in signal 508 and signal fiber 502 are spectrally analyzed, for time period 506 where motion artifacts are present, resulting in spectra 608 and 602 respectively. Frequency ranges 606 are found at which spectrum 608 is comparable in amplitude to spectrum 602. Those frequency components of spectrum 602 are filtered out, resulting in filtered spectrum 612. Since the fluctuations in light intensity due to Doppler shifts associated with blood flow tend to be broader in frequency than the fluctuations due to motion artifacts, a corrected spectrum 614, in frequency ranges 606, is estimated from the amplitude of spectrum 612 at neighboring frequencies. The amplitude of corrected spectrum 614, integrated over a broad range of frequencies, is used instead of spectrum 602 to determine a corrected blood flow rate. Spectrum 614 generally gives a more reliable measure of blood flow rate than would be obtained by simply subtracting spectrum 608 from spectrum 602, since the absolute amplitude of the motion artifact contribution to spectrum 602 may be very different from the absolute amplitude of the motion artifact contribution to spectrum 608.

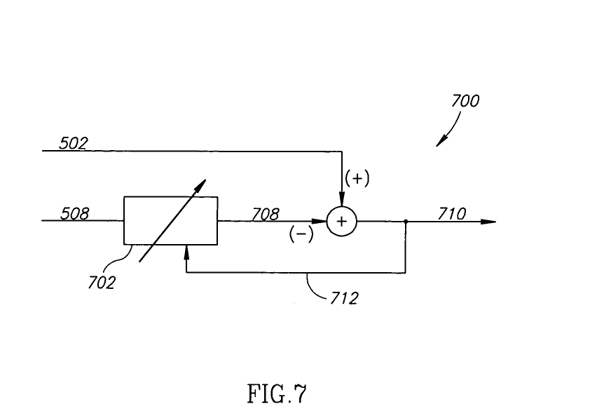

In some embodiments of the invention, an adaptive filtering method, illustrated in block diagram 700 in FIG. 7, is used to reduce the effect of laser noise, thereby increasing the signal to noise ratio of the Doppler blood flow measurement. Such adaptive filtering may be used when the same laser 403, or any light source, is used to provide illuminating light for both signal fiber 410 and monitoring fiber 418, as shown in FIGS. 4A and 4B. In this case, the laser noise in signal 502 from signal fiber 410 is correlated with the laser noise in signal 508 from monitoring fiber 418, although the amplitude of the laser noise may differ in signals 502 and 508.

As shown in FIG. 7, signal 508 is fed into an adaptive filter 702 which amplifies or attenuates signal 508 by an adjustable factor, producing an output signal 708. Signal 708 is subtracted from signal 502, to produce an output signal 710. Signal 710 is fed back into adaptive filter 702, by a feedback loop 712, and the adjustable amplifying factor in adaptive filter 702 is adjusted to minimize the fluctuation level of signal 710. The feedback algorithm used is optionally any of many adaptive filter algorithms known to the art. Suitable algorithms are described, for example, on the page “Noise Cancellation (or Interference Calculation),” in the online product documentation of The Mathworks, Inc., 1994-2005, [retrieved on 2005-12-11], retrieved from the Internet: <URL: http://www.mathworks.com/access/helpdesk/ help/toolbox/filterdesign/adaptiv7.html>, the disclosure of which is incorporated herein by reference. Because the laser noise in signal 502 is correlated with the laser noise in signal 508, this procedure is expected to produce a signal 710 with the laser noise substantially reduced. If laser noise (as opposed to detector noise, for example) is the dominant noise in signal 502, then signal 710 will have a substantially higher signal to noise ratio than signal 502, and signal 710 can be used to make a more accurate measurement of blood flow than signal 502.

Reducing the laser noise in signal 502 by adaptive filtering is especially useful if laser 403 is a gas laser, for example an ultraviolet gas laser, since gas lasers typically have a rather high level of normal relative intensity noise, between 1% and 3%. But even if laser 403 is a single mode semiconductor laser, which typically has a normal relative intensity noise level of about 0.5%, the adaptive filtering method may improve the signal to noise ratio of the Doppler blood flow measurement. Because noise levels in lasers vary in time, filtering out the noise may give a more accurate and stable measure of blood flow than attempting to compensate for noise by applying a correction, that is constant in time, to the fluctuation level in signal 502.

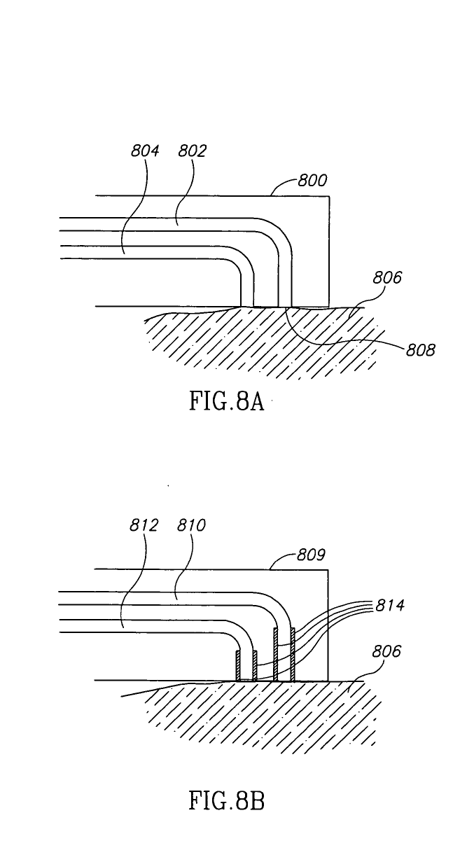

FIG. 8A shows an axial cross-sectional view of a probe body 800, similar to probe body 106 or probe body 400 for example. An illuminating fiber 802, and a nearby receiving fiber 804, have ends in contact with body tissue 806, for example the wall of the urethra. A portion of the light traveling down illuminating fiber 802 toward body tissue 806 may reflect internally from distal end 808 of fiber 802, and a portion of the reflected light may be received by receiving fiber 804, without ever going through body tissue 806. This “cross-talk” light in fiber 804 may interfere with the informative light signal in fiber 804 coming from body tissue 806. Although cross-talk is particularly a problem near the ends of fibers, it can occur elsewhere in fibers as well. Cross-talk is generally believed to be worse for polymer fibers, which are characterized by high numerical aperture and often have no buffer layer, than for silica fibers, which have lower numerical aperture and are normally used with a protective buffer layer made of polyamide or other materials.

FIG. 8B shows a probe body 809, similar to probe body 800, with two optical fibers 810 and 812 similar to fibers 802 and 804 in FIG. 8A. However, fibers 810 and 812 are optionally each coated on their radial surface with a layer of light-blocking material 814, which blocks light that would otherwise scatter out of fiber 810 into fiber 812. Light-blocking material 814 thus prevents or reduces cross-talk between the fibers. In some embodiments of the invention, the light-blocking material coats only one of the fibers. Optionally, the light-blocking material is present only near the ends of the fibers, where cross-talk is particularly likely due to light reflected from the surface of the fiber end. Alternatively, the light-blocking material coats a greater portion of the length of one or more fibers, but less than half of the length, on their radial surfaces.

Optionally, light-blocking material 814 absorbs light. For example it comprises a material, that substantially absorbs the wavelength or wavelengths of light transmitted by fiber 810. Additionally or alternatively, the light-blocking material reflects light, particularly the wavelengths of light transmitted by fiber 810. Optionally, light-blocking material 814 is a glue or a potting material, and may also serve to hold fiber 810 and/or fiber 812 in place in probe 809. Optionally, light-blocking material 814 is a paint.

The invention has been described in the context of the best mode for carrying it out. It should be understood that not all features shown in the drawing or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. An optical probe, for acquiring measurements of material in a surface, the probe comprising:
a probe body;
at least one illuminating optical fiber that transmits light to a distal end thereof to illuminate a region of the surface and interact with the material; and
at least one receiving optical fiber, positioned to receive light that has been transmitted by the illuminating fiber to the region and has interacted with the material, which received light is used for acquiring the measurements, the receiving fiber thereby being defined as associated with the illuminating fiber;
wherein at least one of the fibers has a portion inside the probe body with a bend.

2. An optical probe according to claim 1, wherein the probe body is less than 3 mm in diameter.

3. An optical probe according to claim 1, wherein the bend is sufficiently sharp so that light of a wavelength used for acquiring the measurements is attenuated by at least 5% when passing through the bend.

4. An optical probe according to claim 1, wherein the bend has a mean radius of curvature, over at least one 20 degree segment, of less than 5 times the fiber diameter.

5. An optical probe according to claim 1, wherein the probe body comprises a structure which holds a portion of said at least one of the fibers, including the bend, rigidly in place with respect to the probe body.

6. An optical probe according to claim 1, wherein the probe has a longitudinal axis, and the portion of the fiber inside the probe lies substantially along the longitudinal axis proximal to the bend, and the bend orients the distal end of the fiber to face away from the axis.

7. An optical probe, according to claim 6 wherein the distal end faces along a direction more than 45 degrees from the longitudinal axis.

8. An optical probe according to claim 7, wherein the distal end faces along a direction more than 80 degrees from the longitudinal axis.

9. An optical probe according to claim 6, wherein the at least one illuminating fiber and the at least one receiving fiber both have portions that lie substantially along the longitudinal axis inside the probe body, and end in a bend that orients the distal end facing away from the axis.

10. An optical probe according to claim 9, wherein the distal ends face directions more than 45 degrees from the longitudinal axis.

11. An optical probe according to claim 9, wherein the distal ends face directions more than 80 degrees from the longitudinal axis.

12. A method of acquiring optical data of material in a surface, the method comprising:
placing an optical probe according to claim 6 against the surface, with the longitudinal axis substantially parallel to the surface, and the distal ends of the at least one illuminating optical fiber and the at least one receiving optical fiber in optical contact with the surface;
illuminating a region of the surface with light through the at least one illuminating optical fiber; and
generating the data responsive to light received from the region of the surface by the at least one receiving optical fiber.

13. A method according to claim 12, wherein placing the probe against the surface comprises holding the probe manually, without mechanically fixing the probe in place with respect to the surface.

14. A method according to claim 12, wherein the surface comprises a surface of an internal organ of the body, the method also including:
surgically exposing the internal organ; and
leaving the probe in place against the surface, to monitor the internal organ when is the organ is no longer exposed.

15. An optical probe according to claim 1, wherein the material is human or animal tissue and the surface is a wall of a lumen inside the human or animal.

16. An optical probe according to claim 1, wherein at least one of the optical fibers is a polymer optical fiber.

17. An optical probe according to claim 1, wherein the at least one receiving optical fibers comprise two receiving optical fibers, associated with one of the at least one illuminating optical fibers.

18. An optical probe according to claim 1, wherein the at least one illuminating optical fiber comprises at least two illuminating optical fibers.

19. An optical probe according to claim 18, wherein the at least two illuminating optical fibers have distal ends the centers of which are between 2.5 and 5 mm apart.

20. An optical probe according to claim 18, wherein the at least two illuminating optical fibers have distal ends the centers of which are at least 3.5 mm apart.

21. An optical probe according to claim 18, wherein the distal ends of the at least two illuminating optical fibers are more than 5 times as far apart as the penetrating distance in the material in the surface, of the most penetrating light of the illuminating light that interacts with the surface material.

22. An optical probe according to claim 18, wherein the light transmitted by the at least two illuminating optical fibers is used to acquire measurements of a same parameter of the material, and the at least two illuminating optical fibers have distal ends spaced apart at a distance over which variations in said parameter are substantially uncorrelated.

23. An optical probe according to claim 1, wherein the center of the distal end of the at least one receiving optical fiber is located at a distance from the center of the distal end of the at least one illuminating optical fiber that it is associated with, equal to less than two times a penetrating distance, in the material in the wall, of the least penetrating light of the illuminating light that interacts with the material.

24. A urinary catheter comprising a probe according to claim 1, the catheter adapted so that the probe is positioned to acquire measurements of the wall of the urethra, when the catheter is in place in the urethra.

25. A urinary catheter according to claim 24, comprising at least one opening in its side, through which a distal portion of the illuminating fiber and a distal portion of the receiving fiber extend, such that the illuminating fiber and receiving fiber are optically coupled with the wall of the urethra when the catheter is in place in the urethra.

26. An optical probe according to claim 1, wherein the bend in the fiber is machined out of a volume of the fiber material, and thereby has relatively low internal stress.

27. A system comprising:
an optical probe according to claim 1; and
a light source, coupled to the proximal end of the at least one illuminating optical fibers, which source produces the light for acquiring the measurements, between 315 nm and 525 nm.

28. An optical probe, for acquiring measurements of a material, the probe comprising:
a plurality of optical fibers adapted for transmitting light to and from the material to acquire said measurements; and
a light-blocking material, covering at least a portion but less than 50% of at least one of the optical fibers, that reduces optical crosstalk between the fibers.

29. An optical probe according to claim 28, wherein the light-blocking material reduces optical crosstalk by absorbing light.

30. An optical probe according to claim 28, wherein the light-blocking material reduces optical crosstalk by reflecting light.

31. An optical probe according to claim 28, wherein the light-blocking material mechanically couples said optical fiber to the probe or to another optical fiber or to both.

32. An optical probe according to claim 28, wherein the probe comprises a probe body having a longitudinal axis, and wherein an optical fiber of the plurality of optical fibers has a portion that lies substantially along the longitudinal axis and ends in a bend that orients a distal end of the fiber facing away from the longitudinal axis, and the portion of the fiber covered by the light-blocking material is between the bend and the distal end.

33. An optical probe system for measuring blood flow in a tissue region, the system comprising:
a first optical circuit that provides light that interacts with the tissue and generates a first signal indicative of the blood flow in the tissue region, responsive to the interacting light; and
a second optical circuit that generates a second signal that indicates when the first signal is affected by a motion artifact.

34. An optical probe system according to claim 33, wherein the light is coherent, and the first signal indicates blood flow by a variance in Doppler shifts.

35. An optical probe system according to claim 34, wherein the first optical circuit comprises an illuminating optical fiber that transmits the light to the tissue region and a receiving signal optical fiber that receives the light the interacts with the tissue.

36. An optical probe system according to claim 35 wherein the second optical circuit comprises a receiving monitoring optical fiber that receives light that has not interacted with the tissue.

37. An optical probe system according to claim 36, wherein the illuminating optical fiber has a bend, and the light received by the receiving monitoring optical fiber leaks out of the illuminating optical fiber at the bend.

38. An optical probe system according to claim 36 wherein the receiving optical fibers are constrained to move together, so that motion of the receiving signal optical fiber which causes a motion artifact in the first optical circuit also causes a motion artifact in the second optical circuit.

39. An optical probe system according to claim 38, wherein the second optical circuit also comprises an illuminating monitoring optical fiber, constrained to move with the illuminating optical fiber of the first optical circuit, which transmits the light received by the receiving monitoring optical cable.

40. An optical probe system according to claim 36, also comprising:
a light source that provides the light transmitted by the first optical circuit to the tissue region, and the light received by the second optical circuit; and
an adaptive filter, adapted to filter the first signal, using the second signal, to produce a filtered first signal with reduced light source noise compared to the unfiltered first signal.

41. An optical probe system according to claim 33, also comprising a filter, adapted to filter the first signal, using the second signal, to produce a filtered first signal with reduced motion artifacts compared to the unfiltered first signal.

42. An optical probe for acquiring measurements of material in a surface, the probe comprising:
a plurality of illuminating optical fibers that transmit light to illuminate spatially separated regions of the surface and to interact with the material in the regions;
a set of at least one receiving optical fiber associated with each of the illuminating optical fibers, each receiving fiber positioned to receive at least a portion of the light that has interacted with the material in the region illuminated by the associated illuminating fiber; and
an interface to a detector for each region, to convert light received from each region to a separate signal.

43. A system for acquiring optical measurements of material in a surface, the system comprising:
an optical probe according to claim 42;
a detector for each set of receiving fibers, which converts light received from each region into a signal for the region; and
a controller adapted to analyze the signals to produce a local measurement result from each region, and to use the local measurement results to produce the measurement, disregarding or giving less weight to aberrant local measurement results.
